(19) United States
(12) Patent Application Publication
(10) Pub. No.: US 2022/0107091 A1
(43) Pub. Date: Apr. 7, 2022

(54) HEATING COOKER SYSTEM AND DEODORIZATION METHOD

(71) Applicant: Shinpo Co., Ltd., Nagoya-shi (JP)

(72) Inventors: Toshiaki Tanaka, Seto (JP); Tomoya Asakawa, Kariya (JP)

(21) Appl. No.: 17/139,528

(22) Filed: Dec. 31, 2020

(30) Foreign Application Priority Data
Oct. 2, 2020 (JP) 2020-167430

(51) Int. Cl. F24C 15/20; A47J 36/38; B01D 39/20; B01D 46/00; F24C 15/32; A62C 3/00; A47B 33/00; A61L 9/014

(52) U.S. Cl. CPC F24C 15/2042; A47J 36/38; B01D 39/2027; B01D 46/0023; F24C 15/2035; A61L 2209/22; A62C 3/006; A47B 33/00; A61L 9/014; A61L 2209/14; F24C 15/322

(57) ABSTRACT

Provided is a compact heating cooker system capable of discharging steam and smoke generated when heating and cooking food. A heating cooker system 1A includes a table 10 and a heating cooker 20. The heating cooker 20 includes a casing 21 having an exhaust port 21a, a heat source 22 for heating a cooking utensil X, a top plate 23 having a plurality of suction holes 23a in communication with the exhaust port 21a, an exhaust passage 27 in communication with the exhaust port 21a, a filter 24 arranged inside the exhaust passage 27, an exhaust fan 25 arranged downstream of the filter 24, and a deodorizing device 26 arranged downstream of the exhaust fan 25.

### PRIORITY CLAIM

This application claims the benefit of Japan Patent Application No. 2020-167430 filed Oct. 2, 2020, the contents of which are incorporated herein by reference.

### FIELD OF THE INVENTION

The present invention relates to a heating cooker system for heating and cooking food. In addition, the present invention relates to a deodorization method using this heating cooker system.

### BACKGROUND OF THE INVENTION

Conventionally, when a heating cooker is used, smoke and steam that is generated when heating food is discharged by an exhaust device. Japanese Unexamined Patent Application Publication No. 2002-085262 “(Patent Literature 1”) discloses a heating cooker including a duct 25. The duct 25 passes under the floor and extends to the outside of a building. A suction fan is provided in the middle of the duct 25, and by driving the suction fan, smoke generated when heating and cooking is discharged to the outside of the building through the duct 25.

In a case where an exhaust duct needs to be provided under the floor as in Patent Literature 1, significant construction work is required to provide the exhaust duct. Further, since the heating cooker must be installed in a position that fits the exhaust duct, the position at which the heating cooker can be installed is restricted.

The present invention provides a compact heating cooker system that is capable of discharging steam and smoke. The present invention further provides a deodorization method using the heating cooker system.

### SUMMARY OF THE INVENTION

The present invention has the following features.

### Item 1

A heating cooker system comprising a table having a tabletop and a housing arranged below the tabletop, and a heating cooker arranged inside the table,

wherein the heating cooker comprises:

a casing including a bottom plate and side plates, the bottom plate having an exhaust port, a cooking utensil being placeable in a space surrounded by the side plates and the bottom plate;

a heat source configured to heat the cooking utensil;

a top plate arranged above the casing and having a plurality of suction holes in communication with the exhaust port;

an exhaust passage in communication with the exhaust port;

a filter arranged inside the exhaust passage;

an exhaust fan arranged downstream of the filter; and

a deodorizing device arranged downstream of the exhaust fan.

### Item 2

The heating cooker system according to Item 1, wherein the cooking utensil is a pot.

### Item 3

A heating cooker system comprising a table having a tabletop and a housing arranged below the tabletop, and a heating cooker arranged inside the table,

wherein the heating cooker comprises:

an outer casing including a bottom plate having an exhaust port and side plates;

an inner casing arranged inside the outer casing, a cooking utensil being placeable in the inner casing;

a heat source configured to heat the cooking utensil;

a top plate arranged above the outer casing and the inner casing and having a plurality of suction holes in communication with the exhaust port;

an exhaust passage in communication with the exhaust port;

a filter arranged inside the exhaust passage;

an exhaust fan arranged downstream of the filter; and

a deodorizing device arranged downstream of the exhaust fan.

### Item 4

The heating cooker system according to Item 3, wherein the cooking utensil is a net or a grate.

### Item 5

The heating cooker system according to any one of Items 1 to 4, wherein the top plate includes an upper plate and side plates, and the suction holes are provided in the side plates.

### Item 6

The heating cooker system according to any one of Items 1 to 5, wherein the heating cooker includes a plurality of the filter, the plurality of filters including a first filter that removes at least fat, and a second filter arranged downstream of the first filter and removes at least fat particles smaller than the fat removed by the first filter.

### Item 7

The heating cooker system according to any one of Items 1 to 6, wherein the exhaust fan includes an intake port and a discharge port, the exhaust fan being arranged so that the discharge port is oriented downwardly.

### Item 8

The heating cooker system according to Item 7, wherein the exhaust fan is arranged so that the discharge port is positioned above the deodorizing device.

### Item 9

The heating cooker system according to any one of Items 1 to 8, wherein the exhaust fan includes a motor, the exhaust fan being arranged so that a motor shaft of the motor is horizontal.

### Item 10

The heating cooker system according to any one of Items 1 to 9, wherein the heating cooker includes a plurality of the deodorizing device, the plurality of deodorizing devices being arranged on a same plane.

### Item 11

The heating cooker system according to any one of Items 1 to 10, wherein only one exhaust port is provided at a position offset from a center of the bottom plate, the heating cooker further including a baffle arranged immediately before the exhaust port to partially cover the exhaust port.

### Item 12

The heating cooker system according to any one of Items 1 to 11, wherein the heating cooker further includes an excessive temperature rise detector and a fire damper arranged upstream of the deodorizing device and configured to open or close the exhaust passage, the excessive temperature rise detector being configured to cause the fire damper to shut off when a temperature in the heating cooker exceeds a predetermined temperature.

### Item 13

The heating cooker system according to Item 12, wherein the excessive temperature rise detector is a thermal fuse.

### Item 14

A method for deodorizing a space in which the heating cooker system is installed, by performing an idle operation of the heating cooker system according to any one of Items 1 to 13.

### Effects of the Invention

In the heating cooker system according to the present invention, a heating cooker equipped with an exhaust mechanism is arranged inside a table. Thus, a compact heating cooker system can be provided. In addition, since there is no need for an exhaust duct to be installed under the floor, the heating cooker system can be moved to any location.

By using the heating cooker system according to the present invention, the air in the space where the heating cooker system is installed can be effectively deodorized.

### BRIEF DESCRIPTION OF THE DRAWINGS

Preferred and alternative examples of the present invention are described in detail below with reference to the following drawings:

FIG. 1 shows a perspective view of a heating cooker system according to a first embodiment of the present invention.

FIG. 2 shows a cross-sectional view taken along line II-II of FIG. 1.

FIG. 3(a) shows a perspective view of a top plate, and FIG. 3(b) shows a cross-sectional view taken along line of FIG. 3(a).

FIG. 4 shows a cross-sectional view that schematically illustrates an exhaust flow in the heating cooker system according to the first embodiment.

FIG. 5 shows a cross-sectional view of a heating cooker system according to a second embodiment of the present invention.

FIG. 6 shows a perspective view of a heating cooker system according to a third embodiment of the present invention.

FIG. 7 shows a cross-sectional view taken along line VII-VII of FIG. 6.

FIG. 8 shows a cross-sectional view of a heating cooker system according to a fourth embodiment of the present invention.

### DETAILED DESCRIPTION OF THE PREFERRED EMBODIMENT

Heating cooker systems 1A to 1D according to the present invention will be described with reference to the drawings. The heating cooker systems 1A to 1D according to the present invention are devices for heating and cooking food. After describing the construction and features of the heating cooker systems 1A to 1D, a deodorization method using the heating cooker systems 1A to 1D will be described.

### A First Embodiment

### A-1 Construction of Heating Cooker System

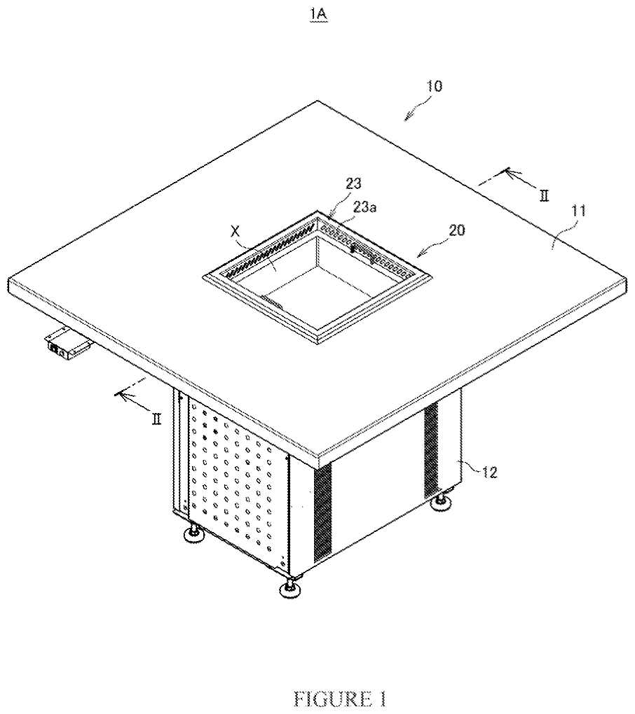

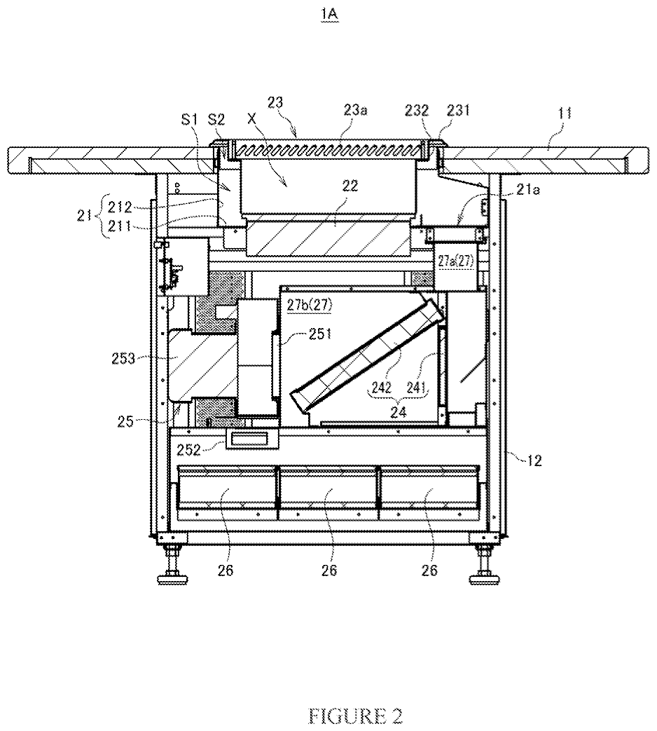

First, with reference to FIGS. 1 to 4, a heating cooker system 1A according to a first embodiment will be described. FIG. 1 is a perspective view of the heating cooker system 1A. FIG. 2 is a cross-sectional view taken along line II-II of FIG. 1.

The heating cooker system 1A according to the first embodiment is a heating cooker system for pot cooking. “Pot cooking” as used here refers to cooking in which liquid (e.g., broth) and foodstuffs are put inside a pot and heated.

In one embodiment, the heating cooker system 1A is a heating cooker system for shabu-shabu. “Shabu-shabu” is Japanese pot cooking in which thin slices of meat are cooked in a broth heated in a pot, and is eaten with dipping sauce.

In one embodiment, the heating cooker system 1A is a heating cooker system for yosenabe. “Yosenabe” is Japanese pot cooking in which foodstuffs such as meat, seafood, vegetables, tofu, etc. are stewed in a broth.

In one embodiment, the heating cooker system 1A is a heating cooker system for mizutaki nabe. “Mizutaki nabe” is Japanese pot cooking in which chicken and vegetables are put in a pot full of water and boiled, and is eaten with dipping sauce.

In one embodiment, the heating cooker system 1A is a heating cooker system for hinabe. “Hinabe” is Chinese pot cooking using a spicy broth containing several types of spices such as pepper fruits, etc., in which foodstuffs such as meat, fish, and vegetables are stewed.

The heating cooker system 1A may also be used for pot cooking other than the pot cooking listed above.

The heating cooker system 1A includes a table 10 and a heating cooker 20. The table 10 includes a tabletop 11 and a housing 12 arranged under the tabletop 11. At the center of the tabletop 11 is provided a rectangular opening. The heating cooker 20 is arranged inside the table 10. The heating cooker 20 includes a casing 21, a heat source 22, a top plate 23, a filter 24, an exhaust fan 25, and a deodorizing device 26. In the heating cooker system 1A a pot X is used as a cooking utensil X, which pot X is heated by the heat source 22.

The pot X is arranged inside the casing 21 and above the heat source 22. The top plate 23 has a plurality of suction holes 23a, and is arranged above the pot X. The casing 21 is provided with only one exhaust port 21a. The exhaust port 21a is connected to the exhaust fan 25 via an exhaust passage 27. The exhaust passage 27 consists of a vertical passage 27a extending downwardly from the exhaust port 21a and a horizontal passage 27b that extends horizontally from the vertical passage 27a. The filter 24 is arranged inside the horizontal passage 27b. The deodorizing device 26 is arranged downstream of the exhaust fan 25. At a bottom of the housing 12 is provided an opening in communication with the deodorizing device 26.

When the exhaust fan 25 is driven, steam from liquid evaporating inside the pot X is sucked through the suction holes 23a of the top plate 23. Steam sucked through the suction holes 23a flows through the exhaust port 21a into the exhaust passage 27. Steam flowing in the exhaust passage 27 is filtered by the filter 24, deodorized by the deodorizing device 26, and then discharged from the bottom of the housing 12.

Below, each component will be described in detail. It should be noted that in the below description, “steam” may be referred to as “exhaust”.

As shown in FIG. 2, the casing 21 includes a rectangular bottom plate 211 and four side plates 212 rising up from the bottom plate 211. The exhaust port 21a is provided at a peripheral section of the bottom plate 211 (i.e., at a position offset from the center of the bottom plate 211). As described above, only one exhaust port 21a is provided at one location of the peripheral section of the bottom plate 211. The pot x is placed in the space surrounded by the bottom plate 211 and the side plates 212. The pot X is arranged such that its side walls are spaced apart from the side plates 212 of the casing. Accordingly, when the pot X is placed in the casing 21, there is a gap S1 between the side walls of the pot X and the side plates 211.

As shown in FIG. 2, the heat source 22 is arranged at the bottom plate 211 of the casing 21. The heat source 22 is, for example, an induction heater, an electric heater, or a gas burner. The heat source 22 heats the pot X which is arranged either in contact with the heat source 22 or spaced apart from the heat source 22.

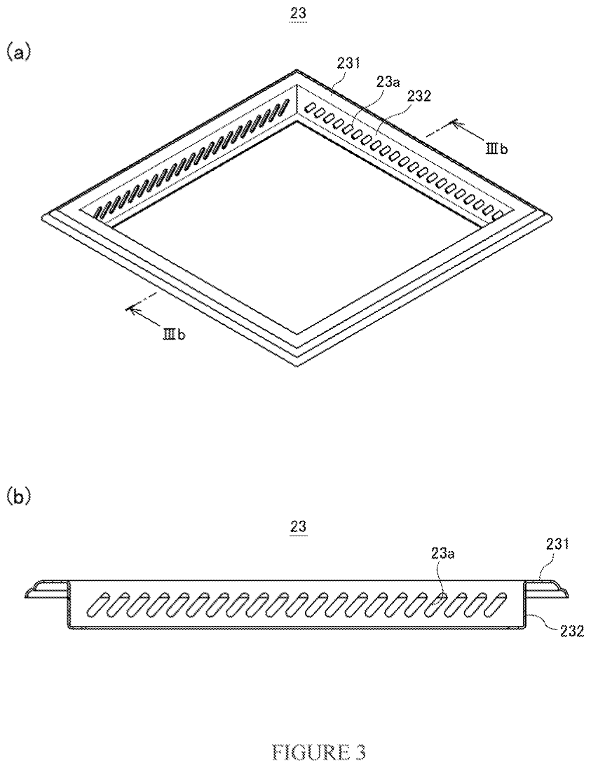

FIG. 3(a) is a perspective view of the top plate 23, and FIG. 3(b) is a cross-sectional view taken along line IIIb-IIIb of FIG. 3(a). The top plate 23 is rectangular or annular, and includes an upper plate 231 and side plates 232 having a plurality of suction holes 23a. The upper plate 231 is placed on the tabletop 11 of the table T. The side plates 232 extend downwardly from the upper plate 231. When the top plate 23 is placed on the tabletop 11, the side plates 232 are spaced apart from the side surfaces forming the opening of the tabletop 11. That is to say, when the top plate 23 is placed on the tabletop 11, there is a gap S2 (see FIG. 2) between the side plates 232 and the side surfaces forming the opening of the tabletop 11. This gap S2 is in communication with the gap S1. During operation of the exhaust fan 25, steam from the pot X is sucked through the suction holes 23a, and flows into the exhaust port 21a through the gap S2 and the gap S1.

As shown in FIG. 2, the filter 24 is arranged inside the horizontal passage 27b and between the exhaust port 21a and the exhaust fan 25. The filter 24 is a grease filter that removes at least fat contained in the steam. In addition to fat, the filter 24 may be capable of removing odorous components and food particles. In one embodiment, the heating cooker 20 includes a plurality of filters 24. In one embodiment, the heating cooker 20 includes a first filter 241 and a second filter 242 arranged downstream of the first filter 241. The steam flowing through the exhaust passage 27 is filtered by the first filter 241, and is then further filtered by the second filter 242. The first filter 241 is intended to remove relatively large fat particles from the steam, and the second filter 242 is intended to remove fat particles smaller than those removed by the first filter 241. In one embodiment, the first filter 241 is made of, for example, a porous nickel-aluminum alloy, is detachable from the heating cooker 20, and may be washed and reused.

As shown in FIG. 2, the exhaust fan 25 is a device for sucking the steam generated by liquid in the pot X and discharging the steam to the outside of the table 10. The exhaust fan 25 is arranged downstream of the filter 24 and upstream of the deodorizing device 26. The exhaust fan 25 includes an intake port 251, a discharge port 252, and a motor 253. The exhaust fan 25 is arranged so that the discharge port 252 is oriented downwardly and the motor shaft of the motor 253 is horizontal. Further, the exhaust fan 25 is arranged so that its entirety is above the deodorizing device 26, and the discharge port 252 is arranged directly above the deodorizing device 26. Accordingly, exhaust from the exhaust fan 25 is discharged toward the deodorizing device 26 from above the deodorizing device 26.

As shown in FIG. 2, the deodorizing device 26 is arranged downstream of the exhaust fan 25. The deodorizing device 26 includes at least a deodorizing material, and deodorizes the steam that has passed through the exhaust fan 25. The deodorizing material is, for example, activated carbon. In one embodiment, the deodorizing device 26 includes a deodorizing material and a grease filter, and both deodorizes and removes fat from the steam that has passed through the exhaust fan 25. In one embodiment, the heating cooker 20 includes a plurality of deodorizing devices 26. The plurality of deodorizing devices 26 are lined up on the same plane.

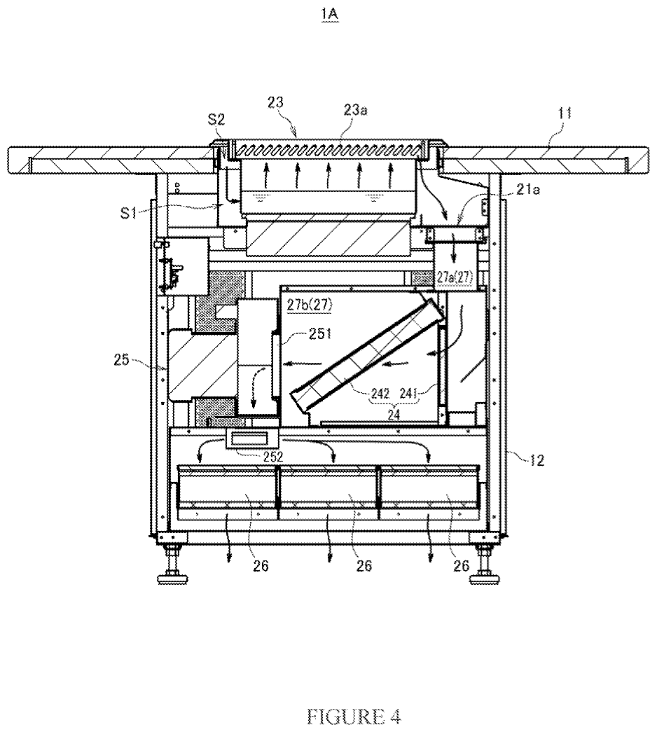

Referring to FIG. 4, the flow of steam during use of the heating cooker system 1A will be described. In FIG. 4, the flow of steam is schematically illustrated by arrows.

During use of the heating cooker system 1A, steam generated from liquid in the pot X is sucked through the suction holes 23a of the top plate 23 by the exhaust action (suction action) of the exhaust fan 25. The steam sucked through the suction holes 23a flows into the exhaust port 21a through the gap S2 and the gap S1, and from the exhaust port 21a enters the exhaust passage 27. The steam that has entered the exhaust passage 27 is filtered by the filter 24, and is then sucked into the exhaust fan 25. Then, the steam is discharged toward the deodorizing device 26 from above the deodorizing device 26, and, after being deodorized by the deodorizing device 26, is discharged from the bottom of the housing 12 (table 10).

### A-2 Features of the Heating Cooker System

In the heating cooker system 1A, the heating cooker 20 including an exhaust mechanism is housed within the housing 12 of the table 10. Therefore, the heating cooker system 1A according to the present invention is compact.

By having the exhaust passage 27 include not only the vertical passage 27a, but also the horizontal passage 27b in communication with the vertical passage 27a, the length of the exhaust passage 27 can be increased. This makes it possible to increase the amount of fat adhering to the inner walls of the exhaust passage 27, thereby reducing the amount of fat in the steam passing through the filter 24. As a result, the lifespan of the filter 24 can be extended.

By having the heating cooker 20 include a plurality of filters 24, and having the first filter 241 remove comparatively large fat particles, the lifespan of the second filter 242 can be extended.

By arranging the exhaust fan 25 downstream of the filter 24, the amount of fat adhering to the exhaust fan 25 can be reduced. This prevents deterioration of exhaust efficiency of the exhaust fan 25.

In embodiments where the exhaust fan 25 is arranged downstream of the deodorizing device 26, pressure loss occurs due to the deodorizing device 26, which necessitates an increased output of the exhaust fan 25 in order to avoid deterioration of the suction force due to the pressure loss. This tends to make the system noisy. However, by arranging the exhaust fan 25 upstream of the deodorizing device 26 as in the present invention, pressure loss due to the deodorizing device 26 can be reduced, which allows for a lower output of the exhaust fan 25, and as a result, reduced noise from the exhaust fan 25. Further, since a reduced output of the exhaust fan 25 allows for a slower speed of the exhaust, the exhaust can remain inside the deodorizing device 26 for a longer duration of time, allowing for more effective deodorization. In addition, if the deodorizing material is a sound-absorbing material such as activated carbon, arranging the exhaust fan 25 upstream of the deodorizing device 26 allows the deodorizing device 26 to reduce the amount of noise. Further, by arranging the exhaust fan 25 upstream of the deodorizing device 26, the deodorizing device 26 becomes a resistance that can reduce the speed of the exhaust from the exhaust fan 25, which makes it possible to reduce the speed of the exhaust from the bottom of the table 10.

Since the discharge port 252 of the exhaust fan 25 is oriented downwardly, dirt in the interior of the exhaust fan 25 falls off more easily, which can prevent deterioration of the exhaust efficiency of the exhaust fan 25.

By arranging the exhaust fan 25 so that the motor shaft of the motor 253 is horizontal, vibration of the exhaust fan 25 can be reduced. Further, arranging the exhaust fan 25 so that the motor shaft of the motor 253 is horizontal allows for a wider space below the exhaust fan 25. This makes it possible to arrange more deodorizing devices 26 or a bigger deodorizing device 26 under the exhaust fan 25. As a result, the speed of the exhaust passing through the deodorizing device 26 can be reduced, and the deodorization capabilities of the deodorizing device 26 can be improved.

By arranging the discharge port 252 of the exhaust fan 25 above the deodorizing device 26 to discharge exhaust toward the deodorizing device 26 from above the deodorizing device 26, exhaust can be delivered as evenly as possible to the entire deodorizing device 26. As a result, the deodorization capabilities of the deodorizing device 26 can be improved.

By lining up a plurality of deodorizing devices 26 on the same plane, the deodorization load on each deodorizing device 26 can be equalized. As a result, the lifespan of the deodorizing devices 26 can be extended.

### B Second Embodiment

B-1 Construction of the heating cooker system

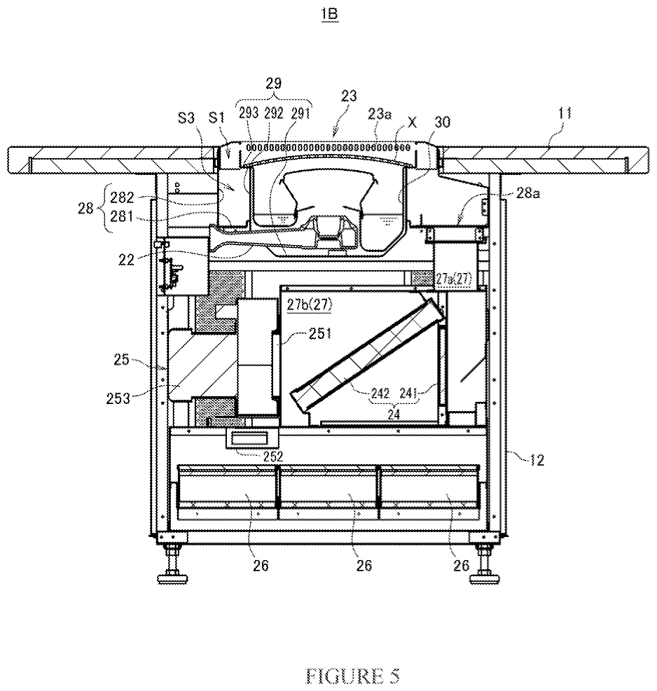

A heating cooker system 1B according to a second embodiment will be described with reference to FIG. 5. FIG. 5 shows a cross-sectional view of the heating cooker system 1B. The heating cooker system 1B is a device for grilling foodstuffs. It should be noted that the following description is focused on the differences from the embodiment described above, and as such components with the same construction or the same functionality as those of the above embodiment are given the same reference numerals as in the above embodiment, and description thereof is omitted.

The heating cooker system 1B includes an outer casing 28, an inner casing 29, a drain pan 30, a heat source 22, a top plate 23, a filter 24, an exhaust fan 25, and a deodorizing device 26. A cooking utensil X in the heating cooker system 1B is, for example, a net or a grate. Further, since the heating cooker system 1B according to the second embodiment is a device for grilling foodstuffs as mentioned above, the exhaust fan 25 is mainly for smoke.

The outer casing 28 includes a rectangular bottom plate 281 and four side plates 282 rising up from the bottom plate 281. An exhaust port 28a is provided at a peripheral section of the bottom plate 281 (i.e., at a position offset from the center of the bottom plate 281). Only one exhaust port 28a is provided at one location of the peripheral section of the bottom plate 281. The exhaust port 28a is connected to the exhaust fan 25 via an exhaust passage 27.

The inner casing 29 includes a rectangular bottom plate 291 and four side plates 292 rising up from the bottom plate 291. At the top of the side plates 292 there is provided a flange 293 on which the cooking utensil X is to be placed. The inner casing 29 is arranged in a space surrounded by the bottom plate 281 and the side plates 282 of the outer casing 28. The inner casing 29 is arranged so that its side plates 292 are spaced apart from the side plates 282 of the outer casing 28. Accordingly, when the inner casing 29 is arranged inside the outer casing 28, a gap S3 is present between the side plates 282 of the outer casing 28 and the side plates 292 of the inner casing 29.

The drain pan 30 is arranged inside the inner casing 29. The center of the drain pan 30 is provided with an opening for the heat source 22. When the heating cooker system 1B is used, water is poured into the drain pan 30.

The cooking utensil X is placed on the flange 293 of the inner casing 29. Foodstuffs are placed on the cooking utensil X.

The heat source 22 is arranged below the cooking utensil X. The heat source 22 is, for example, an electric heater or a gas burner, which heats the cooking utensil X and the foodstuffs placed on the cooking utensil X from below the cooking utensil X.

When the exhaust fan 25 is driven, smoke produced when grilling the foodstuffs is sucked through the suction holes 23a of the top plate 23. The smoke flows through the gap S1 and the gap S3 into the exhaust port 28a, and then flows from the exhaust port 28a into the exhaust passage 27. Smoke flowing in the exhaust passage 27 is filtered by the filter 24, deodorized by the deodorizing device 26, and then discharged from the bottom of the housing 12.

### B-2 Features of the Heating Cooker System

The heating cooker system 1B according to the second embodiment has the same features as the heating cooker system 1A according to the first embodiment.

### C Third Embodiment

### C-1 Construction of the Heating Cooker System

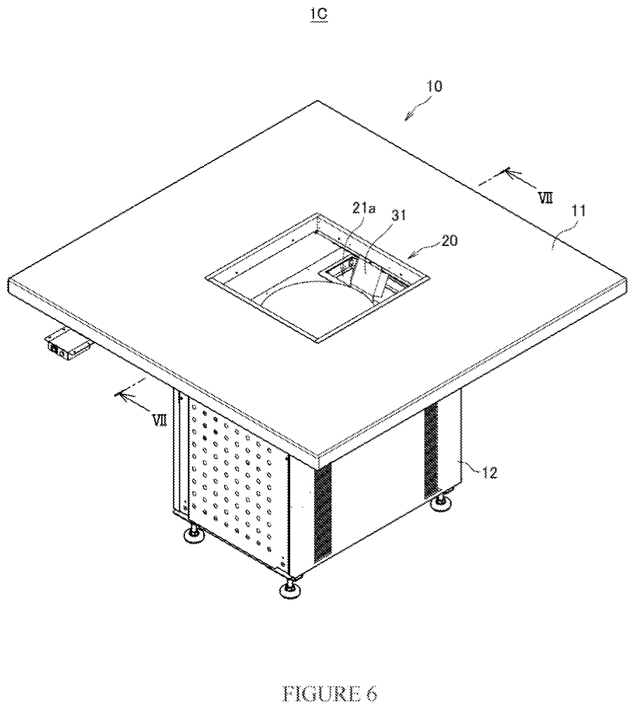

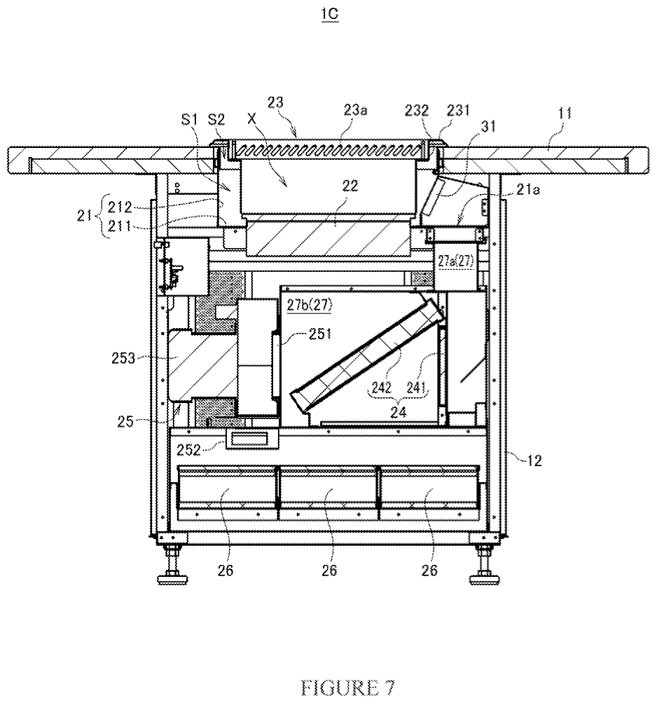

A heating cooker system 1C according to a third embodiment will be described with reference to FIGS. 6 and 7. FIG. 6 shows a perspective view of the heating cooker system 1C. In FIG. 6, some components are omitted to make it easier to see the interior of the heating cooker system 1C. FIG. 7 shows a cross-sectional view of the heating cooker system 1C. It should be noted that the following description is focused on the differences from the embodiments described above, and as such components with the same construction or the same functionality as those of the above embodiments are given the same reference numerals as in the above embodiments, and description thereof is omitted.

In addition to the components of the heating cooker system 1A according to the first embodiment and the heating cooker system 1B according to the second embodiment, the heating cooker system 1C further includes a baffle 31. FIGS. 6 and 7 show the heating cooker system 1A according to the first embodiment with an added baffle 31.

When the exhaust fan 25 is operated in the heating cooker system 1B and the heating cooker system 1B, the exhaust suction force of the plurality of suction holes 23a of the top plate 23 tends to be stronger at the suction holes 23a close to the exhaust port 21a, 28a (exhaust fan 25), and, conversely, tends to be weak at the suction holes 23a far from the exhaust port 21a, 28a (exhaust fan 25). Therefore, when the exhaust fan 25 is operated in the heating cooker systems 1A and 1B, there is a possibility that the suction force acting on the suction holes 23a close to the exhaust port 21a, 28a becomes stronger than necessary, while the suction force acting on the suction holes 23a far from the exhaust ports 21a, 28a becomes insufficient. There is thus a risk of steam or smoke not being sufficiently sucked through the suction holes 23a far from the exhaust port 21a, 28a, which leads to steam or smoke rising from the side far from the exhaust port 21a, 28a, disturbing the eaters' meal. The heating cooker system 1C resolves this problem by providing the baffle 31.

As shown in FIGS. 6 and 7, the baffle 31 is arranged immediately before the exhaust port 21a to partially cover the exhaust port 21a. The baffle is secured to the casing 21 by fastening means such as bolts, etc. In one embodiment, the baffle 31 is arranged to cover the center of the exhaust port 21a.

When adding the baffle 31 to the heating cooker system 1B, the baffle 31 is arranged immediately before the exhaust port 28a to partially cover the exhaust port 28a. The baffle is secured to the casing 28 by fastening means such as bolts, etc. In one embodiment, the baffle 31 is arranged to cover the center of the exhaust port 28a.

### C-2 Features of the Heating Cooker System

By having the baffle 31 arranged immediately before the exhaust port 21a, 28a, the suction force acting on the suction holes 23a close to the exhaust port 21a, 28a is suppressed. As a result, the suction force acting on the suction holes 23a far from the exhaust port 21a, 28a becomes relatively stronger. In other words, the suction force across all of the suction holes 23a becomes more equalized, so that steam or smoke is sucked in equally by the suction holes 23a close to the exhaust port 21a, 28a and the suction holes 23a far from the exhaust port 21a, 28a. This prevents steam or smoke from rising at the side far from the exhaust port 21a, 28a.

### D Fourth Embodiment

### D-1 Construction of the Heating Cooker System

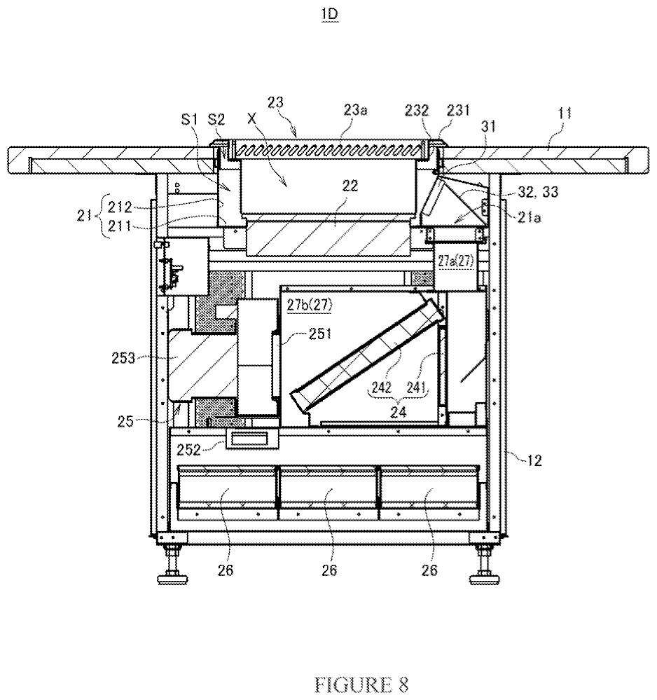

A heating cooker system 1D according to a fourth embodiment will be described with reference to FIG. 8. FIG. 8 shows a cross-sectional view of the heating cooker system 1D. It should be noted that the following description is focused on the differences from the embodiments described above, and as such components with the same construction or the same functionality as those of the above embodiments are given the same reference numerals as in the above embodiments, and description thereof is omitted.

In addition to the components of the heating cooker system 1A according to the first embodiment, the heating cooker system 1B according to the second embodiment, and the heating cooker system 1C according to the third embodiment, the heating cooker system 1D further includes an excessive temperature rise detector 32 and a fire damper 33. FIG. 8 shows the heating cooker system 1C according to the third embodiment with an added excessive temperature rise detector 32 and fire damper 33. A deodorizing device 26 of the heating cooker system 1D includes a flammable deodorizing material such as activated carbon.

The heating cooker system 1D has prevents a flammable deodorizing material from being burned by hot exhaust in a case where, for example, a malfunction of the heat source 22 causes an abnormally high temperature of the heat source 22.

The fire damper 33 is arranged upstream of the deodorizing device 26, and is configured to close or open the exhaust passage 27. In one embodiment, the fire damper 33 is arranged immediately before the exhaust port 21a, and shuts off the exhaust port 21a during an abnormal operation of the heat source 22. The excessive temperature rise detector 32 operates the fire damper 33 to close the fire damper 33 when the temperature in the heating cooker 20 becomes equal to or higher than a preset temperature.

In one embodiment, the excessive temperature rise detector 32 is a thermal fuse. When the temperature in the heating cooker 20 becomes equal to or higher than a predetermined temperature, the excessive temperature rise detector (thermal fuse) 32 fuses, causing a drive mechanism of the fire damper 33 to operate, so that the fire damper 33 shuts off the exhaust port 21a.

### D-2 Features of the Heating Cooker System

When the temperature in the heating cooker 20 becomes equal to or higher than a predetermined temperature, the fire damper 33 shuts off the exhaust passage 27, so that hot exhaust can be prevented from flowing into the exhaust passage 27. This prevents hot exhaust from reaching the deodorizing device 26, so that the burning of the flammable deodorizing material can be prevented.

### E Deodorization Method

Described below is a method using the heating cooker system 1A to 1D to deodorize the air in a space where the heating cooker system 1A to 1D is installed.

Since the heating cooker system 1A to 1D includes the exhaust fan 25 and the deodorizing device 26, the air in the room where the heating cooker system 1A to 1D is installed can be deodorized by operating the exhaust fan 25 in a state where no foodstuffs are placed in or on the cooking utensil X. This method of using the heating cooker system 1A to 1D is useful as a method for deodorizing the interior of a restaurant after business hours.

When deodorizing, the heat source 22 is preferably not operated. In addition, deodorization is preferably carried out with the cooking utensil X removed from the heating cooker 20.

### F Variant

The above embodiments describe preferred embodiments of the present invention. The present invention is not limited to the above embodiments, but various modifications may be made without departing from the scope and spirit of the invention. The present invention may, for example, employ the variant shown below.

The opening in the tabletop 11 of the table 10 may be circular. In this case, the bottom plate 211 and side plates 212 of the casing 21, the bottom plate 281 and side plates 282 of the outer casing 28, and the bottom plate 291 and the side plates 292 of the inner casing 29 may be circular, and the top plate 23 may be annular.

### REFERENCE SIGNS LIST

1A to 1D Heating cooker system
10 Table
11 Tabletop
12 Housing
20 Heating cooker
21 Casing
21a Exhaust port
211 Bottom plate
212 Side plate
22 Heat source
23 Top plate
23a Suction hole
231 Upper plate
232 Side plate
24 Filter
241 First filter
242 Second filter
25 Exhaust fan
251 Intake port
252 Discharge port
253 Motor
26 Deodorizing device
27 Exhaust passage
28 Outer casing
28a Exhaust port
281 Bottom plate
282 Side plate
29 Inner casing
31 Baffle
32 Excessive temperature rise detector
33 Fire damper
X Cooking utensil (pot, net, grate)

While the preferred embodiment of the invention has been illustrated and described, as noted above, many changes can be made without departing from the spirit and scope of the invention. Accordingly, the scope of the invention is not limited by the disclosure of the preferred embodiment. Instead, the invention should be determined entirely by reference to the claims that follow.

1. A heating cooker system comprising a table having a tabletop and a housing arranged below the tabletop, and a heating cooker arranged inside the table,
wherein the heating cooker comprises:
a casing including a bottom plate and side plates, the bottom plate having an exhaust port, a cooking utensil being placeable in a space surrounded by the side plates and the bottom plate;
a heat source configured to heat the cooking utensil;
a top plate arranged above the casing and having a plurality of suction holes in communication with the exhaust port;
an exhaust passage in communication with the exhaust port;
a filter arranged inside the exhaust passage;
an exhaust fan arranged downstream of the filter; and
a deodorizing device arranged downstream of the exhaust fan.

2. The heating cooker system according to claim 1, wherein the cooking utensil is a pot.

3. A heating cooker system comprising a table having a tabletop and a housing arranged below the tabletop, and a heating cooker arranged inside the table,
wherein the heating cooker comprises:
an outer casing including a bottom plate having an exhaust port and side plates;
an inner casing arranged inside the outer casing, a cooking utensil being placeable in the inner casing;
a heat source configured to heat the cooking utensil;
a top plate arranged above the outer casing and the inner casing and having a plurality of suction holes in communication with the exhaust port;
an exhaust passage in communication with the exhaust port;
a filter arranged inside the exhaust passage;
an exhaust fan arranged downstream of the filter; and
a deodorizing device arranged downstream of the exhaust fan.

4. The heating cooker system according to claim 3, wherein the cooking utensil is a net or a grate.

5. The heating cooker system according to claim 1, wherein the top plate includes an upper plate and side plates, and the suction holes are provided in the side plates.

6. The heating cooker system according to claim 1, wherein the heating cooker includes a plurality of the filter, the plurality of filters including a first filter that removes at least fat, and a second filter arranged downstream of the first filter and configured to remove at least fat particles smaller than the fat removed by the first filter.

7. The heating cooker system according to claim 1, wherein the exhaust fan includes an intake port and a discharge port, the exhaust fan being arranged so that the discharge port is oriented downwardly.

8. The heating cooker system according to claim 7, wherein the exhaust fan is arranged so that the discharge port is positioned above the deodorizing device.

9. The heating cooker system according to claim 1, wherein the exhaust fan includes a motor, the exhaust fan being arranged so that a motor shaft of the motor is horizontal.

10. The heating cooker system according to claim 1, wherein the heating cooker includes a plurality of the deodorizing device, the plurality of deodorizing devices being arranged on a same plane.

11. The heating cooker system according to claim 1, wherein only one exhaust port is provided at a position offset from a center of the bottom plate, the heating cooker further including a baffle arranged immediately before the exhaust port to partially cover the exhaust port.

12. The heating cooker system according to claim 1, wherein the heating cooker further includes an excessive temperature rise detector and a fire damper arranged upstream of the deodorizing device and configured to open or close the exhaust passage, the excessive temperature rise detector being configured to cause the fire damper to shut off when a temperature in the heating cooker exceeds a predetermined temperature.

13. The heating cooker system according to claim 12, wherein the excessive temperature rise detector is a thermal fuse.

14. A method for deodorizing a space in which the heating cooker system is installed, by performing an idle operation of the heating cooker system according to claim 1.
